(19) United States
(12) Patent Application Publication
(10) Pub. No.: US 2025/0134125 A1
(43) Pub. Date: May 1, 2025

(54) METHOD FOR THE REMOVAL OF ACRYLAMIDE FROM FOODS AND STIMULANTS

(71) Applicants: ANKA ANGEWANDTE KAFFEETECHNOLOGIE GMBH, Bremen (DE); C-LECTA GMBH, Leipzig (DE)

(72) Inventors: Nikolina PANTELIC, Breman (DE); Meike HOLTMANN, Bremen (DE); Oliver SÜSSE-HERRMANN, Hude (DE); Gregor HOFFMANN, Markkleeberg (DE); Stefan SCHÖNERT, Leipzig (DE)

(21) Appl. No.: 18/682,841

(22) PCT Filed: Aug. 12, 2022
(86) PCT No.: PCT/EP2022/072660

§ 371 Date: Feb. 9, 2024

(30) Foreign Application Priority Data
Aug. 12, 2021 (WO) PCT/EP2021/072477

(51) Int. Cl. A23F 5/16; C12N 9/80

(52) U.S. Cl. CPC A23F 5/163; C12N 9/80; C12Y 305/01004

(57) ABSTRACT

The invention relates inter alia to the field of the production of foods and stimulants, more particularly coffee products and coffee substitute products having a reduced acrylamide content. The invention provides a method for removing acrylamide from various food matrices, more particularly from a coffee or coffee substitute product matrix, by using a biocatalyst to degrade acrylamide.

### TECHNICAL FIELD

The present invention is in the field of the production of foodstuffs and luxury foods, in particular coffee and coffee substitute products with reduced acrylamide content. A method for removing acrylamide from various food matrices, in particular from a coffee or coffee substitute product matrix, is provided which involves the use of a biocatalyst to degrade acrylamide.

### BACKGROUND OF THE INVENTION

The demands of end consumers for foodstuffs and luxury foods that are consistently safe and safe to consume are constantly high and place special requirements on producers. A new EU regulation from 2018 (EU 2017/2158) classifies acrylamide as a process contaminant and potential health risk for end consumers. As a result, food and luxury food producers are obliged to keep the acrylamide content in foodstuff and luxury foods below a certain level or to reduce it. In animal experiments, acrylamide has a carcinogenic and mutagenic effect. Acrylamide is formed in all frying, baking and deep-frying processes of carbohydrate-containing raw materials and is the product of the heating of asparagine with reducing sugars (e.g. glucose and fructose) as part of the so-called Maillard reaction.

In the specific case of the production of coffee and coffee substitutes, these processes involve the brewing or extraction of roasted and ground coffee beans and their substitutes, such as chicory, barley or rye. During roasting, the coffee beans are typically subjected to temperatures between 145° C. and 250° C., during which complex chemical reactions, the Maillard reaction, caramelization and pyrolysis take place. Through these reactions, the chemical, physical and sensory properties of the roasted products are changed, which are fundamentally important for the taste of the end product. In addition, other substances are formed or released that are important for the end product, such as antioxidants (Jin et al. “Relationship between antioxidants and acrylamide formation: A review”, Food Research International, 2013, p. 611-620). Acrylamide is also formed here as an undesirable method contaminant through the roasting of coffee and coffee substitute products (Anese, M. “Acrylamid in Coffee and Coffee Substitutes, Acrylamid in Food, 2016, p. 181-195). The guideline values for acrylamide in the new EU regulation are 400 μg/kg for roasted coffee, 850 μg/kg for soluble coffee, 500 μg/kg for coffee substitutes made exclusively from grain and 4000 μg/kg for products made from chicory.

Acrylamide is also produced in a variety of (other) methodes in the foodstuff and luxury food industry. For example, acrylamide is produced when potatoes are deep-fried. It is advantageous or even necessary to partially or completely remove this from the semi-finished or finished product, in particular to meet the requirements of the EU Regulation (EU 2017/2158) on the one hand and to obtain a safe end product that is harmless to the consumer on the other.

A large number of processes exist which describe the removal of acrylamide from a preparation using enzymes. For example, the international application WO 2004/083423 A1 discloses a method for the degradation of acrylamide in a preparation by using an amidase. The European application EP 0272025 A2 also relates to a method for the decomposition of acrylamide using an amidase.

Furthermore, the international patent application WO 2021/148508 discloses a method for the degradation of acrylamide in a preparation using a particularly thermo-and pH-stable amidase. The application does not disclose a separation of the aqueous preparations by membrane methodes in order to treat only a partial stream with an enzyme.

All methods described are such methods in which the enzyme used remains in the preparation.

In order to achieve a safe foodstuff or luxury food for the consumer, the enzymes used are usually inactivated by heat at the end of the manufacturing process and are no longer present in their active form. The enzyme used, also known as biocatalyst, can therefore not be reused. Even though enzymes can be produced inexpensively nowadays, large quantities of enzyme are required, especially in the large-scale production of foodstuff and luxury foods, so that reusing the used enzyme is desirable not only for cost reasons but also for reasons of sustainability.

For example, the international application WO 2016/004949 A1 describes a method for producing a coffee product comprising an enzyme treatment of coffee extract in which the enzyme used in the preparation is thermally inactivated or retained via a membrane. The enzymatic treatment and subsequent post-treatment takes place in the coffee extract. In addition to aromatic substances, this also contains e.g. particulate components, which are (also) important for the sensory properties of the final coffee product. When the enzyme used is separated via a membrane, these important components are separated and are no longer present in the end product. The international applications WO 2007/011531 A1 and WO 2016/207384 A1 also disclose the retention of an enzyme so that it is not inactivated and can be reused. Here, the enzyme treatment takes place in the coffee extract and the particulate components are also separated during a possible membrane separation. Furthermore, all of the above-mentioned applications disclose carbohydrate-degrading enzymes for the production of coffee preparations. None of these applications disclose a method for the removal of acrylamide.

The international application WO 2013/005145 describes the enzymatic removal of the acrylamide precursors asparagine and aspartate before roasting the beans. The described method comprises an extraction of the beans with water, enzymatic treatment of the aqueous extract using asparaginase and aspartase, the concentration of the extract, the mixing of the concentrated extract with the treated green beans and subsequent drying. Only subsequently, the extract is roasted and further processed, whereby less acrylamide is formed during the roasting method as the reactants are missing. The method is characterized by additional water and energy consumption as well as the additional equipment required in the upstream step of aqueous extraction and enzymatic treatment and is not very sustainable overall. With this method, there is a risk that extracted components, the return of which is necessary for the production of a coffee with perfect taste and sensory properties, are lost through concentration or drying, as components can be discharged via the necessary waste water and exhaust air flow, thus changing the organoleptic properties of the end product. In contrast, the method according to the invention can be easily integrated into the existing methods of instant coffee production and does not include an outlet or waste streams through which sensory or flavor-important components could be discharged.

The application WO 2021/123163 describes a method for producing a liquid coffee concentrate with a reduced acrylamide content, wherein acrylamide is separated from a first weakly aromatic aqueous coffee extract by means of a selectively permeable membrane, whereby a second weakly aromatic aqueous coffee extract with a lower acrylamide content than the first weakly aromatic aqueous coffee extract is formed, and the second weakly aromatic aqueous coffee extract is subsequently combined with a strongly aromatic aqueous coffee extract. An enzymatic treatment of one of the aqueous coffee extracts is not provided.

During enzymatic treatment of preparations which are consumed or used primarily for their taste or odor, it is important to preserve all substances and components during the enzyme treatment which contribute to the aroma, taste or odor in order to obtain a final product which is free from taste and olfactory defects. Methods for the enzymatic treatment of preparations as well as hydrolysis reactions as disclosed in the applications described above may have the disadvantage that when such enzymes are used, side reactions or overreactions may also occur which, for example, convert or degrade the desired components of such preparations. Furthermore, with the methods known from the prior art, there is a risk of undesirable separation of other components that are important for the sensory properties of the end product, particularly if the enzymes used for treatment are inactivated or removed. This results in an end product that differs in its properties from the desired end product.

It was thus the primary task of the present invention to provide a method which provides a safe end product with a reduced acrylamide content and at the same time preserves the substances and components necessary for the desired flavor and olfactory properties of the desired end product, preferably without the enzymes used to reduce acrylamide remaining in the end product. Furthermore, the method is to be applied economically on a large scale.

### DESCRIPTION OF THE INVENTION

This primary task is solved according to the present invention by providing a method for removing or converting acrylamide from an aqueous preparation to produce a final product with reduced acrylamide content, comprising the following steps:
(i) providing an aqueous preparation comprising acrylamide and other components as a starting product for the production of a final product with a reduced acrylamide content;
(ii) dividing the aqueous preparation into two material streams to obtain a retentate (R1) and a permeate (P1), wherein the retentate (R1) contains the further components (K1), and wherein the permeate (P1) contains acrylamide and preferably no or a small fraction of further components (K2);
(iii) bringing the permeate (P1) into contact with an acrylamide-reducing biocatalyst and obtaining a permeate (P2) with a reduced acrylamide content;
(iv) combining the obtained permeate (P2) having a reduced content of acrylamide and the retentate (R1) from step (ii) and obtaining an aqueous preparation having a reduced content of acrylamide, wherein the permeate (P2) does not contain a biocatalyst;
(v) optionally, further processing the aqueous preparation with reduced acrylamide content from step (iv) to obtain a final product.

An aqueous preparation in the context of the present invention is preferably a preparation which has a water content of >50% by weight, preferably >70% by weight and particularly preferably more than >90% by weight, based on the total weight of the preparation.

Preferred aqueous preparations to be used in the context of the present invention are coffee or coffee substitute extracts in unconcentrated or concentrated form, fruit juices or fruit preparations, lemonades, tea extracts, milk or milk product preparations and pulpy masses, preferably for the production of breakfast cereals.

“Coffee extracts” in the context of the present invention are extracts of roasted and ground coffee beans.

“Coffee substitute extracts” in the context of the present invention are extracts of roasted and ground substitutes of coffee beans, such as chicory, barley, lupine, ginseng, reishi, spelt, corn, dandelion root or rye.

The acrylamide contained in the aqueous preparation can be the method product of the Maillard reaction, as described above. Acrylamide (prop-2-enamide) has a molecular weight of 71.08 g/mol or 71.08 Da.

Furthermore, the aqueous preparation contains components that fulfill a function as flavorants or odorants of the preparation. Furthermore, certain components contained in the aqueous preparation can contribute to a pleasant, desirable mouthfeel of the preparation.

The term components in the context of the present invention refers to substances other than acrylamide, which may be either dissolved or present as particles. Particles in the sense of the invention are both suspended particles (particles in the actual sense) and colloidal or dispersed dissolved particles. Particles can also be other dissolved substances such as flavorings, polysaccharides such as lignin, proteins or fats.

Components within the meaning of the invention refer to all particles and dissolved substances provided that they correspond to the respective particle or component size according to the invention. The particles present may have an average particle size of less than 50 mm, preferably less than 5 mm, particularly preferably in the range of 0.3-100 μm. Preferably, the present particles can have a particle size in the range of 1-100 μm. They are of fundamental importance for the mouthfeel of the product. In coffee and coffee substitute preparations in particular, these substances give the end product its characteristic smell and taste and should therefore be retained.

The particles, especially polysaccharides and proteins, present in a used aqueous preparation can be degraded by enzymatic or thermal hydrolysis reactions. The present invention provides a method by means of which components, i.e. particles and dissolved substances, of a certain size can be separated and protected from undesired enzymatic or thermal hydrolysis reactions.

The components present in the aqueous preparation used are referred to as (K1) and (K2). The components K1 are particles and dissolved substances with a size greater than 0.01 μm, preferably greater than 0.1 μm, preferably greater than 1 μm, further preferably greater than 10 μm, preferably greater than 100 μm and further preferably greater than 500 μm. The components (K2) are all particles and dissolved substances with a size smaller than 0.01 μm, preferably smaller than 0.1 μm, further preferably smaller than 1 μm, preferably smaller than 10 μm and further preferably smaller than 100 μm. The used aqueous preparation can be separated into two material streams containing either (K1) or (K2). Combining the material streams in step (iv), which contain the components (K1) and (K2), results in a mixture which, with the exception of the acrylamide and the reaction products of the acrylamide, corresponds to the aqueous preparation originally used.

The terms permeate and retentate in the context of the present invention describe the material flows obtained after separation of an initial mixture via a physical method. The retentate is the part of the initial mixture that is retained during this method and the permeate is the part of the initial mixture that is not retained.

A preferred physical method for producing a permeate and a retentate is the separation of a material flow by means of a membrane. In a preferred embodiment, a permeate obtained from an initial mixture by means of a first physical separation can be further divided into a further retentate and a further permeate by means of a second physical separation. In a preferred embodiment, the separation parameters of the first physical separation differ from the separation parameters of the second physical separation. In an alternative preferred embodiment, the separation parameters of the first physical separation and the second physical separation do not differ.

The composition of (K1) and (K2) is determined by the type and pore size of a first membrane (M1). The material flow of the permeate is driven by a pressure gradient or a concentration gradient, or by a combined pressure and concentration gradient.

The particles and dissolved substances of the components (K1) remain in the retentate (R1) and are not present in the permeate (P1). Preferably, the remaining components (K1) are not brought into contact with the biocatalyst. It is further preferred in the context of the present invention that the components (K1) are particulate or dissolved substances and/or are such that fulfill certain sensory properties in the final product, such as imparting or contributing to a pleasant mouthfeel. The components (K2) have a smaller size, preferably a size comparable to acrylamide, and are present in the permeate (P1) after separation of the material streams in step (ii). It is preferred in the context of the present invention that the components (K2) are recycled into the aqueous preparation in step (iv) after contacting the permeate (P1) with the biocatalyst and transferring it into the permeate (P2) in step (iii). Furthermore, it is preferred that the components (K2) are smaller than the biocatalyst.

“Dividing the material flows” in the context of the present invention describes the splitting of the aqueous preparation into a remaining part, the so-called retentate (R1), in which the additional, remaining components (K1) are located and into a part permeate (P1), in which, in addition to water, acrylamide and possibly a small proportion of dissolved further components (K2) with a similar size or with a similar molecular weight to acrylamide are located. Preferably, the permeate (P1) is free or essentially free of other components which are undissolved particles. The composition of the components (K2) in the permeate (P1) results from the type and pore size of the membrane (M1) used.

In a preferred embodiment, the type and pore size of the membrane (M1) are selected such that acrylamide can pass through the membrane (M1). In a further preferred embodiment, the type and pore size of the membrane (M1) are selected such that the biocatalyst cannot pass through the membrane (M1).

“Bringing into contact” in the context of the present invention means that the acrylamide contained in the permeate (P1) comes into contact with the biocatalyst and can thus be converted by it. Preferably, the conversion in the context of the present invention takes place by hydrolysis, so that harmless acrylic acid is formed. The resulting material stream after enzyme treatment (permeate (P2)) has a reduced acrylamide content. Accordingly, an “acrylamide-reducing biocatalyst” in the context of the present invention refers to a biocatalyst which enzymatically converts or converts acrylamide into another product and thus reduces or reduces the content or concentration of acrylamide.

Preferably, in the context of the present invention, the permeate (P2) is obtained by separating the material stream from the biocatalyst after contacting the permeate (P1) with the biocatalyst in step (iii) and before combining the permeate (P2) and the retentate R1 in step (iv). The separation of the biocatalyst is preferably carried out by means of separation, e.g. centrifugation, sedimentation, or filtration via a sieve, via a mesh or via a membrane. Particularly preferred in the context of the present invention is the separation of the biocatalyst via a membrane (M2).

The retentate (R2) obtained during the separation of the biocatalyst contains the biocatalyst and the permeate (P2) contains no biocatalyst.

In a preferred embodiment of the present invention, steps (iii) and (iv) of the method according to the invention comprise the following substeps:
(iii-1) contacting the permeate (P1) with an acrylamide-reducing biocatalyst and obtaining a permeate (P2) having a reduced content of acrylamide, the permeate (P2) comprising the biocatalyst;
(iii-2) separating the biocatalyst from the permeate (P2) from step (iii-1) by means of separation over a membrane (M2) and obtaining a retentate (R2) containing the biocatalyst and obtaining the permeate (P2) with a reduced content of acrylamide, wherein the permeate (P2) does not comprise any biocatalyst;
(iv) combining the resulting permeate (P2) with reduced content of acrylamide from step (iii-2) and the retentate (R1) from step (ii) and obtaining an aqueous preparation with reduced content of acrylamide.

In a preferred embodiment of the present invention, steps (ii) and (iii-2) of the method according to the invention are carried out via two different membranes (M1) and (M2), wherein the type and pore size of the membranes (M1) and (M2) may differ and are selected such that the biocatalyst cannot pass through the membrane (M1).

In a preferred embodiment of the present invention, steps (ii) and (iii-2) of the method according to the invention are carried out via a single membrane (M1), the type and pore size of the membrane (M1) being selected such that the biocatalyst cannot pass through the membrane (M1).

In a preferred embodiment of the invention, the pore size of the membrane (M2), which should retain the biocatalyst, is smaller than the size of the biocatalyst. In a particularly preferred embodiment, the pore size of the membrane (M2) is smaller than the biocatalyst by a factor of 1.5. It is further preferred that the pore size of the membrane is smaller than the biocatalyst by a factor of 2, it is even more preferred if the pore size of the membrane is smaller than the biocatalyst by a factor of 2.5 and most preferred if the pore size of the membrane is smaller than the biocatalyst by a factor of 3 or more.

In a preferred embodiment, the biocatalyst is separated from the permeate (P2) as described above via a membrane (M2) which has a pore size which is larger than the size of the components (K2) contained in the permeate (P2), and smaller than the components (K1) contained in the retentate (R1), and smaller than the biocatalyst.

In a further preferred embodiment, the pore sizes of the membrane (M1) are smaller than or equal to the pores of the membrane (M2).

In a further preferred embodiment, the pore sizes of the membrane (M1) are smaller than or equal to the pores of the membrane (M2), and the pore sizes of the membrane (M1) and membrane (M2) are each smaller than the biocatalyst.

It is especially preferred in the context of the present invention if the separation of the biocatalyst is carried out via a membrane with a pore size of d₉₀<100 kDa, preferably d₉₀<50 kDa, particularly preferably d₉₀≤30 kDa, particularly preferably d₉₀≤10 kDa.

In step (iv) of the method according to the present invention, the resulting permeate (P2) having a reduced content of acrylamide and containing the components (K2) is added to the retentate (R1) containing the further components (K1), so that an aqueous preparation having a reduced content of acrylamide compared to the content of acrylamide of the aqueous preparation used from step (i) is obtained.

Preferably, the steps of the method are carried out iteratively, particularly preferably continuously, until the desired reduction in acrylamide has been achieved.

In a preferred embodiment, steps (i), (ii), (iii-1), (iii-2), and (iv) of the method according to the invention are carried out iteratively, particularly preferably continuously, over two membranes (M1) and (M2) until the desired reduction of acrylamide has been achieved.

In a further preferred embodiment, steps (i), (ii), (iii-1), (iii-2), and (iv) of the method according to the invention are carried out iteratively, particularly preferably continuously, over a membrane (M1) until the desired reduction of acrylamide has been achieved.

In a further preferred embodiment, steps (ii), (iii), and (iv) of the method according to the invention are carried out via a single membrane (M1) and comprise the following steps:
(ii) dividing the aqueous preparation into two material streams by means of a membrane (M1) in order to obtain a retentate (R1) and a permeate (P1), wherein the retentate (R1) contains the further components (K1), and wherein the permeate (P1) contains acrylamide and preferably no or a small proportion of further components (K2);
(iii-1) contacting the permeate (P1) with an acrylamide-reducing biocatalyst and obtaining a permeate (P2) having a reduced content of acrylamide, the permeate (P2) comprising the biocatalyst;
(iii-2) separating the biocatalyst from the permeate (P2) from step (iii-1) by means of separation over a membrane (M1) and obtaining a retentate (R2) containing the biocatalyst and obtaining the permeate (P2) with a reduced content of acrylamide, wherein the permeate (P2) does not comprise any biocatalyst;
(iv) combining the resulting permeate (P2) with reduced content of acrylamide from step (iii-2) and the retentate (R1) from step (ii) and obtaining an aqueous preparation with reduced content of acrylamide;
   wherein steps (iii-2) and (iv) are carried out simultaneously.

In a preferred embodiment, steps (ii), (iii-1), (iii-2), and (iv) are carried out continuously across the membrane (M1). Preferably, the separation into two material streams in step (ii) and the combination of permeate P2 and retentate (R1) is carried out by means of diffusion of acrylamide according to its diffusion gradient across the membrane (M1).

In a preferred embodiment of the method according to the invention, the contacting in step (iii) is carried out at ambient temperature, preferably at a temperature of above 30° C., preferably above 40° C., further preferably above 50° C., preferably at a temperature of above 60° C., further preferably at a temperature of above 70° C. and also further preferably at a temperature of above 80° C.

The high temperatures required for enzyme activity and acrylamide degradation can have a negative effect on the components (K1) of the aqueous preparation if the time exceeds 30 minutes. It is therefore particularly preferable to separate these in a first step so that they remain in the retentate (R1) and do not come into contact with elevated temperatures and the biocatalyst.

In a further preferred embodiment of the method according to the invention, the contacting in step (iii) is carried out at a pH in a range from pH 4 to pH 7, preferably in a range from pH 4 to pH 6.5, preferably in a range from pH 4.5 to pH 5.5.

In a preferred embodiment of the method according to the invention, the contacting in step (iii) is carried out for a time of more than 10 minutes, preferably more than 15 minutes, further preferably more than 30 minutes, preferably more than one hour, further preferably more than 2 hours, preferably more than 4 hours, further preferably more than 8 hours, preferably more than 12 hours and particularly preferably more than 16 hours.

The present method can be used to produce an end product that has a reduced acrylamide content, which fulfills the requirements for food stuff and luxury food and preferably meets the provisions of EU Regulation 2017/2158. In addition, the obtained end product advantageously has the same or substantially the same taste and odor profile as a non-treated (coffee) preparation.

In a preferred embodiment of the present invention, the present invention relates to a method according to the present invention, wherein the separation of the material streams in step (ii) is carried out as filtration, preferably as membrane filtration.

During filtration in the context of the present invention, an initial mixture is separated based on the size of the contained particles or components. It should be noted here that the pore size of the filtration material is selected such that the components to be separated remain behind. One form of filtration is membrane filtration, in which a mixture of substances is separated by means of a membrane. In membrane filtration, if the pore size is selected appropriately, both undissolved particles and dissolved components can be separated based on their size or molecular weight. Membrane filtration can, for example, be carried out as co-current or counter-current filtration.

Preferably in the context of the present invention, whenever reference is made to a membrane, the membrane geometry is selected from the group consisting of hollow fiber membrane, hollow fiber membrane module, capillary membrane, capillary membrane module, flat sheet membrane, flat sheet membrane module or plate and frame membrane module, spiral wound membrane module, or tubular or multichannel membrane module.

Further preferred in the context of the present invention, whenever reference is made to a membrane, is the use of at least one membrane material selected from the group consisting of polysulfones, polyethersulfone (PES) cellulose, cellulose esters such as cellulose acetate and cellulose nitrate, regenerated cellulose (RC), silicones, polyamides, polyamidimide, polyamide urea, polycarbonates, ceramics, stainless steel, silver, silicon, zeolite, polyacrylonitrile (PAN), polyethylene (PE), polypropylene (PP), polytetrafluoroethylene (PTFE), polyvinylidene fluoride (PVDF), polyvinyl chloride (PVC) and polypiperazinamide.

Preferably, modified membrane materials as defined above can also be used in the context of the present invention, or a combination of the aforementioned membrane materials and their modified forms.

A further preferred embodiment of the method according to the present invention relates to a method, wherein the separation of the material streams in step (ii) is carried out as membrane filtration and the pore size of the membrane is d₉₀≤100 kDa, preferably d₉₀≤50 kDa, particularly preferably d₉₀≤30 kDa and particularly preferably d₉₀≤10 kDa.

A further preferred embodiment of the method according to the present invention relates to a method, wherein the separation of the material streams in step (ii) is carried out as membrane filtration and the membrane has a pore size in the range from 0.01 to 10 μm, preferably in the range from 0.05 to 5 μm and particularly preferably in the range from 0.1 to 1 μm.

Preferably, the separation of the material streams in step (ii) is carried out by using a membrane M1 with a pore size that is smaller than the size of the remaining components (K1) contained in the retentate (R1) and smaller than the biocatalyst.

Preferably, the biocatalyst is separated before step (iv) by using a membrane (M2) with a pore size that is smaller than the biocatalyst and that is greater than or equal to the components (K2) contained in the permeate (P2).

Preferably, the material streams are split in step (ii) and the process streams are combined in step (iv) by using a membrane M1 with a pore size that is smaller than the size of the biocatalyst with simultaneous separation of the biocatalyst.

The pore size of a membrane can be determined by its do value. This so-called “cut-off” indicates the minimum molecular mass of a globular molecule that is 90% retained by the membrane.

Again, a further preferred embodiment of the method according to the present invention relates to a method according to the present invention, wherein the transmembrane pressure is between 0.01 mbar and 10 bar, preferably between 0.1 mbar and 7.5 bar, further preferably between 1 mbar and 5 bar, particularly preferably between 500 mbar and 2.5 bar, and/or is maintained.

The trans-membrane pressure plays a role in the design of membrane-driven processes. There are basically two different operating modes: Working at a constant transmembrane pressure or at a constant flow rate. In the context of the present invention, it is preferred if the membrane filtration is operated at a continuous flow rate, whereby the transmembrane pressure is within the above-mentioned preferred range.

In a further embodiment of the present method, it is preferred if the membrane filtration takes place at a constant transmembrane pressure and the flow rate is variable.

In a preferred embodiment of the method, the method is carried out using a transmembrane pressure (1) when separating the material streams at membrane (M1) in step (ii), or preferably when separating the biocatalyst in step (iii) via a membrane, using a transmembrane pressure (2) at membrane (M2) during separation of the biocatalyst in step (iii), or using a transmembrane pressure (3) during separation of the material streams via a membrane (M1) in step (ii) and during separation of the biocatalyst via a membrane (M2) in step (iii) of the method according to the invention.

A preferred embodiment relates to a method according to the present invention, wherein the end product is an end product intended for pleasure, nutrition or cosmetic purposes, and is preferably selected from the group comprising fried or deep-fried potato products, corn products, coffee products, coffee substitutes, snacks, wheat products, cosmetics, pastries such as cookies, biscuits, rusks, cereal bars, scones, ice cream wafers, wafers, crumpets, and other baked goods, coffee substitutes, snacks, wheat products, cosmetics, cookies such as cookies, cookies, rusks, cereal bars, scones, ice cream cones, waffles, crumpets, gingerbread, crispbread and bread substitutes, pasta, rice, fish products, meat products, cereals, beer or baby food for children and infants.

Most preferably in the context of the present invention, the end product is a coffee or coffee substitute product.

In the context of the present invention, it is further preferred if the end product is an end product used for animal nutrition, preferably selected from the group consisting of feed, nutrient solutions, nutrient preparations, fruit juices, fruit preparations and milk preparations.

Again, a preferred embodiment relates to a method according to the present invention, wherein the biocatalyst is present as a living microorganism, an inactivated microorganism and/or as a cell lysate and/or as a (partially) purified enzyme and/or as an immobilized enzyme.

In a further preferred embodiment, the enzyme is present as a dimer and/or multimer and/or cross-linked. Methods for the dimerization and multimerization of enzymes are known to the person skilled in the art.

Irrespective of its function, the enzyme may vary in size, e.g. by having polypeptide chains of different lengths or by forming a combination of several enzyme subunits to form an active enzyme. Enzymes which mediate the activity of an amidase according to the invention can consist of individual subunits which can be 15 to 80 kDa in size. Furthermore, there are enzymes which consist of several subunits and can be from 40 to 400,000 kDa in size (e.g. Uniprot P95896, octamer of approx. 55 kDa UE). Enzymes according to the present invention may be naturally present as dimers or multimers or may be prepared by methods known to the skilled person.

A “living microorganism” in the context of the present invention means a microorganism which has the ability to divide or reproduce. This microorganism expresses the biocatalyst as a metabolite.

An “inactivated microorganism” in the context of the present invention refers to a microorganism which lacks the ability to divide or reproduce. The biocatalyst is present inside the microorganism or as a membrane protein or otherwise associated with the membrane of the microorganism and is enzymatically active.

A “cell lysate” in the context of the present invention refers to the product of cell disruption of a microorganism expressing the biocatalyst. As a result of the cell disruption, the microorganism loses its ability to reproduce or divide; the other cell components are detectable in the cell lysate in addition to the biocatalyst.

A “(partially) purified enzyme” in the context of the present invention means an enzyme originating from a cell lysate in which no or only a few cell components are detectable and the content of the enzyme is above 1% by weight, above 2% by weight, above 5% by weight, preferably above 10% by weight, above 15% by weight, above 20% by weight and particularly preferably above 30% by weight.

In a preferred embodiment, the biocatalyst is present as a lyophilizate and is used as such in the method.

In a preferred embodiment, the biocatalyst is present as an immobilized enzyme. Suitable methods for enzyme immobilization include cross-linking, inclusion immobilization, adsorptive or covalent binding to the surface of a carrier material. The contacting in step (iii) of the method according to the present invention is carried out in the case where the biocatalyst is present as an immobilized enzyme by passing the permeate (P1) past the immobilized enzyme so that the acrylamide can react with the enzyme without the enzyme being able to pass into the permeate. The resulting material flow is referred to as permeate (P2) or is equivalent to the permeate (P2) described herein.

In a preferred embodiment, the immobilized enzyme is present as part of a fixed bed or fluidized bed reactor in which the permeate (P1) is brought into contact with the biocatalyst according to step (iii) and transferred into the permeate (P2). Preferably, the acrylamide content changes over the course of the reactor, with the acrylamide content being higher at the beginning of the reactor than at the end of the reactor. This embodiment using a fixed bed reactor is particularly preferred.

In the context of the present invention, it is preferred if the enzyme has a size between 20 and 80 kDa, particularly preferably between 30 and 70 kDa and most preferably between 40 and 60 kDa.

In the context of the present invention, it is particularly preferred if the biocatalyst has a size ratio of from 1:10 to 1:1,000, particularly preferably from 1:10 to 1:100 to the other components (K1) present in the aqueous preparation from step (i).

In a preferred embodiment, the size ratio relative to the diameter of acrylamide to biocatalyst is 1:5 to 1:5,000, further preferably 1:10 to 1:1,000, preferably 1:20 to 1:500 and further preferably 1:50 to 1:100.

A further preferred embodiment of the method according to the present invention relates to a method wherein the biocatalyst is present as a (partially) purified enzyme and/or as an immobilized enzyme which is an amidase, preferably from Pseudonocardia thermophila, Sulfolobus solfataricus, Pyrococcus yayanosii and Sulfolobus tokodaii, particularly preferably from Pseudonocardia thermophila.

Other preferred enzymes for reducing the acrylamide content are amidases as described in the prior art, preferably as described in the applications WO 2004/083423 A1, WO 2006/040345 A2, WO 2012/032472 A2, EP 0272025 A2, WO 2021/1148508 and WO 2021/1148509.

A preferred embodiment relates to a method according to the present invention, wherein the biocatalyst is present as a (partially) purified enzyme and/or as an immobilized enzyme which has an amino acid sequence with a sequence identity of at least 60%, 70%, 80%, 90%, 91%, 92%, 93%, 94%, 95%, 96%, 97%, 98% or 99%, preferably of at least 90%, 91%, 92%, 93%, 94%, 95%, 96%, 97%, 98% or 99%, to the total length of an enzyme having a sequence selected from the group consisting of the sequences according to SEQ ID NO. 1 to SEQ ID NO. 44, particularly preferably selected from the group consisting of the sequences according to SEQ ID NO. 12 to SEQ ID NO. 20 and most preferably selected from the group consisting of the sequences according to SEQ ID NO. 17 to SEQ ID NO. 20.

Whenever the present disclosure refers to sequence identities of amino acid sequences in terms of percentages, these refer to values as they can be calculated using EMBOSS

Water Pairwise Sequence Alignments (Protein) for amino acid sequences. The tools for local sequence alignments provided by the European Molecular Biology Laboratory (EMBL) European Bioinformatics Institute (EBI) use a modified Smith-Waterman algorithm. Furthermore, when performing the respective pairwise alignment of two sequences using the modified Smith-Waterman algorithm, reference is made to the default parameters currently specified by the EMBL-EBI. These are (i) for amino acid sequences: Matrix =BLOSUM62, Gap open penalty=10 and Gap extend penalty=0.5 and (ii) for nucleic acid sequences: Matrix=DNAfull, Gap open penalty=10 and Gap extend penalty=0.5. In addition to the default parameters, when aligning a sequence to be examined (“query sequence”, in EMBOSS the first sequence) with a comparison sequence (“subject sequence”, in EMBOSS the second sequence), the subject sequence must be represented at least 93% over the length of the individual alignment (“sequence coverage” at least 93%); alignments with a lower sequence coverage of the subject sequence are excluded for the determination of sequence identity within the meaning of this application. However, the query sequence may be longer than the length of the alignment, and the sequences of the query sequence represented in the alignment may be above or below 93%.

The term “sequence identity” can therefore be used interchangeably with “sequence homology” in the context of the present invention. This always refers to the total length of an enzyme according to the invention compared to the total length of an enzyme for which the sequence identity or sequence homology is determined.

In a further preferred embodiment, the present invention relates to a method according to the present invention, wherein in step (iii) the permeate (P1) is brought into contact with the biocatalyst by mixing and subsequently this mixture is divided into two material streams to obtain the permeate (P2) and the retentate (R2), wherein the permeate (P2) does not contain a biocatalyst.

Preferably, the permeate (P1) is brought into contact with the biocatalyst by mixing in a sub-step (iii-1) and the subsequent separation of the mixture into two material streams in a sub-step (iii-2) via a membrane (M2).

In a further preferred embodiment, step (iii-2) and step (iv) are carried out simultaneously via a membrane (M2).

In a further preferred embodiment, step (iii-2) and step (iv) are carried out simultaneously via a membrane (M1).

A further preferred embodiment of the method according to the invention relates to a method, wherein the contacting of the biocatalyst in step (iii) with the permeate (P1) takes place in the permeate space of a first membrane module and the combining of the permeate (P2) and the retentate (R1) takes place in the permeate space of a second membrane module and wherein the retentate (R1) is fed into the permeate space of the second membrane module. It is preferred within the scope of the present invention that the method steps are carried out iteratively, preferably continuously, until a desired reduction of acrylamide is achieved.

A further preferred embodiment of the method according to the invention relates to a method, wherein the contacting of the biocatalyst in step (iii) with the permeate (P1) takes place in the permeate space of a first membrane module (M1) and the combining of the permeate (P2) and the retentate (R1) takes place in the permeate space of a second membrane module (M2) and wherein the retentate (R1) is fed into the permeate space of the second membrane module. It is preferred within the scope of the present invention that the method steps are carried out iteratively, preferably continuously, until a desired reduction of acrylamide is achieved.

A further preferred embodiment of the method according to the invention relates to a method, wherein the biocatalyst is brought into contact with the permeate (P1) in the permeate chamber of a first membrane module (M1) in a sub-step (iii-1), in a partial step (iii-2), the biocatalyst is separated from the permeate (P2) by means of a membrane (M2), and in a step (iv), the permeate (P2) and the retentate (R1) are combined in the permeate space of the second membrane module (M2), and the retentate (R1) is fed into the permeate space of the second membrane module. It is preferred within the scope of the present invention that the method steps are carried out iteratively, preferably continuously, until a desired reduction of acrylamide is achieved. Preferably, sub-step (iii-2) and (iv) are carried out simultaneously.

A further preferred embodiment of the method according to the invention relates to a method, wherein the biocatalyst is brought into contact with the permeate (P1) in the permeate space of a first membrane module (M1) in a sub-step (iii-1), in a partial step (iii-2), the biocatalyst is separated from the permeate (P2) by means of the same membrane (M1), and in a step (iv), the permeate (P2) and the retentate (R1) are combined in the retentate space of the first membrane module (M1). It is preferred within the scope of the present invention that the method steps are carried out iteratively, preferably continuously, until a desired reduction of acrylamide is achieved. Preferably, sub-step (iii)-1), (iii-2) and (iv) are carried out simultaneously.

Yet another preferred embodiment relates to a method wherein the permeate (P1) is brought into contact with the biocatalyst via the contact surface of a membrane and wherein the pore size of the membrane is d₉₀≤100 kDa, preferably d₉₀≤50 kDa, particularly preferably d₉₀≤30 kDa and most preferably d₉₀≤10.

In a further preferred embodiment, the biocatalyst is immobilized in the matrix of the membrane. Preferably, the reduction of the acrylamide content and the formation of a permeate (P2) takes place by the permeate (P1) flowing past the contact surface of the membrane and the acrylamide diffusing through the membrane and being converted.

In a preferred embodiment, the conversion of the acrylamide by the immobilized biocatalyst takes place by diffusion through the membrane.

A further preferred embodiment relates to a method according to the present invention, wherein the separation of the material streams according to step (ii) and the contacting of the permeate (P1) with the biocatalyst according to step (iii) are carried out simultaneously via the contact surface of a membrane. It is preferred here if at least one membrane module is used and the biocatalyst is located in the lumen or in the permeate space of this at least one membrane module.

A further preferred embodiment relates to a method according to the present invention, wherein the separation of the material streams according to step (ii) and the contacting of the permeate (P1) with the biocatalyst according to step (iii-1) is carried out simultaneously via the contact surface of a membrane (M1). It is preferred here if at least one membrane module (M1) is used and the biocatalyst is located in the lumen or in the permeate space of this at least one membrane module. It is also preferred here if the separation of the method streams according to step (ii) and the separation of the biocatalyst according to step (iii-2) and the merging of the method streams according to step (iv) take place continuously and the biocatalyst is located in the lumen or in the permeate space of this at least one membrane module (M1).

It is preferred in the context of the present invention if, in addition, contacting the permeate (P1) with the biocatalyst and obtaining a permeate (P2) with a reduced content of acrylamide according to step (iii) and combining the resulting permeate (P2) with a reduced content of acrylamide and the retentate (R1) according to step (iv) are carried out simultaneously via the contact surface of a membrane.

It is preferred in the context of the present invention if, in addition, contacting the permeate (P1) with the biocatalyst and obtaining a permeate (P2) with a reduced content of acrylamide according to step (iii) and combining the resulting permeate (P2) with a reduced content of acrylamide and the retentate (R1) according to step (iv) are carried out simultaneously via the contact surface of a membrane (M2).

It is particularly preferred in the context of the present invention if steps (ii), (iii) and (iv) are carried out continuously over the contact surface of a single membrane. Preferably, the contacting of the biocatalyst with the permeate (P1) and the obtaining of permeate (P2) takes place in the permeate space of a single membrane module. Here, the acrylamide diffuses from the retentate space into the permeate space and comes into contact with the biocatalyst. The substances diffusing from the permeate space back into the retentate space (the so-called permeate (P2)) contain a reduced amount of acrylamide.

It is particularly preferred in the context of the present invention if steps (ii), (iii) and (iv) are carried out continuously over the contact surface of a single membrane (M1) and the contacting of the biocatalyst with the permeate (P1) and the obtaining of permeate P2 takes place in the permeate space of the single membrane module (M1). Here, the acrylamide diffuses from the retentate space of membrane (M1) into the permeate space of membrane (M1) and comes into contact with the biocatalyst. The substances diffusing from the permeate space of the membrane (M1) back into its retentate space (the so-called permeate (P2)) contain a reduced amount of acrylamide.

In such a process design, it is particularly preferred if a hollow fiber module (hollow fiber membrane) is used as the membrane geometry, with the biocatalyst being present in the lumen or permeate space of the hollow fiber module. The aqueous preparation is passed through the hollow fiber module and comes into contact with the biocatalyst via a membrane, whereby the acrylamide diffuses through the membrane and is converted by the biocatalyst. The permeate (P1) and the retentate (R1) are located at the beginning of the hollow fiber module and the permeate (P2) at the end of the hollow fiber module. Furthermore, the flat sheet membrane or laminar stacks are a preferred membrane geometry when using such a method design.

Yet another preferred embodiment relates to a method according to the present invention, wherein the aqueous preparation with reduced acrylamide content obtained in step (iv) has a reduced acrylamide content by at least 20% by weight, preferably by at least 30% by weight, compared to the aqueous preparation provided in step (i). % by weight, further preferably by at least 40% by weight, again preferably by at least 50% by weight, preferably by at least 60% by weight, further preferably by at least 70% by weight, more preferably by at least 80% by weight, particularly preferably by at least 90% by weight, of acrylamide, based on the total weight of the aqueous preparation,
and/or
wherein the aqueous preparation with reduced acrylamide content obtained in step (iv) has an acrylamide content of less than 2000 μg/kg, preferably of less than 850 mg/kg and particularly preferably of less than 500 μg/kg based on the dry mass of the preparation.

A further preferred embodiment relates to a method according to the present invention, wherein the aqueous preparation obtained in step (iv) is further processed in step (v) and wherein the further processing comprises at least one method step selected from the group consisting of drying, evaporating, concentrating, freeze-drying, fluidized bed drying, spray-drying, granulating, comminuting and filtering.

Yet another preferred embodiment relates to a method according to the present invention, wherein steps (ii) and (iii) are repeated, preferably continuously.

Further described herein is an apparatus for removing acrylamide from an aqueous preparation consisting of or comprising
(i) a storage container in which the aqueous preparation is provided,
(ii) a first membrane module, preferably with a membrane (M1),
(iii) a second storage container in which the biocatalyst is provided,
(iv) a second membrane module, preferably with a membrane (M2),
   wherein the first membrane module and the second membrane module have an identical pore size.

A preferred embodiment relates to a device wherein both membrane modules have a pore size of d₉₀≤100 kDa, preferably d₉₀≤50 kDa, particularly preferably d₉₀≤30 kDa and most preferably d₉₀≤10 kDa.

Also described herein is a device for removing acrylamide from an aqueous preparation, consisting of or comprising
(i) a storage container in which the aqueous preparation is provided,
(ii) a first membrane module, preferably with a membrane (M1),
(iii) a second storage container in which the biocatalyst is provided,
(iv) a second membrane module, preferably with a membrane (M2),
   where the first membrane module and the second membrane module have different pore sizes.

A preferred embodiment relates to a device, wherein the first of the two membrane modules has a pore size of d₉₀≤100 kDa, preferably d₉₀≤50 kDa, more preferably d₉₀≤30 kDa and most preferably d₉₀≤10 kDa, and wherein the second of the two membrane modules has a pore size of d₉₀≤100 kDa, preferably d₉₀≤50 kDa, particularly preferably d₉₀≤30 kDa and most preferably d₉₀≤10 kDa. Preferably, the pore size of the first of the two membrane modules is smaller than the size of the pore size of the second of the two membrane modules. In a particularly preferred embodiment, the pore sizes of the two membrane modules are smaller than the biocatalyst.

Also described herein is an apparatus for removing acrylamide from an aqueous preparation consisting of or comprising
(i) a reservoir in which the aqueous preparation is provided,
(ii) a first membrane module, preferably with a membrane (M1),
(iii) a second storage container in which the biocatalyst is provided,
   whereby the method streams are divided and combined via the first membrane module.

Further described herein is an apparatus for removing acrylamide from an aqueous preparation consisting of or comprising
(i) a storage container in which the aqueous preparation is provided,
(ii) a membrane module, preferably a hollow fiber membrane module,
   wherein the membrane module has a lumen in which a biocatalyst is present.

The invention is characterized below by illustrative, non-limiting examples.

### Examples

### Example 1

Raw coffee of the variety Brazil Grinder was roasted to a color value of 110 scale parts (Neuhaus Neotec, Colortest II). The acrylamide content in the roasted coffee was analyzed and found to be 560 μg/kg. 40 kg of roasted coffee were extracted at an extraction temperature of 85° C., resulting in a total yield of 220 kg of coffee extract. One part of the coffee extract was used for the subsequent experiments and one part was used for the freeze concentration to produce the coffee concentrate. The unconcentrated coffee extract had a dry residue of 1.4% and an acrylamide content of 28 μg/L, and the concentrate had a dry residue of 27.5% and an acrylamide content of 630 μg/L.

The unconcentrated coffee extract with a dry residue of 1.4 wt. % was separated into two material streams, permeate (P1) and retentate (R1), via a membrane (hollow fiber module, cut-off: 10 kDA) made of polyethersulfone. The permeate (P1) had an acrylamide content of 23 μg/L, which was increased to 223 μg/L by adding acrylamide, and was then fed into a receiver tank and treated with 6500 U/L amidase. The reaction was carried out at 70° C. The permeate (P1) with the amidase was then split into two material streams via another membrane at a flow rate of 2 mL/min. The resulting permeate (P2) contains no amidase.

The reduction of acrylamide in the permeate (P2) is calculated from the enzyme-treated samples compared to the reference ((P1) with 200 μg/L acrylamide added, without enzyme addition). The acrylamide reduction is constant at 80-90% for a process time of 30-300 minutes.

The terms dry residue and solids content have the same meaning.

### Example 2

The test setup from example 1 was used for continuous operation and the obtained permeate (P2) was returned to the original coffee extract. The acrylamide reduction after 60-120 minutes was 84-93%.

### Example 3

In a further experiment, the coffee extract produced in example 1 was treated in a diffusion membrane reactor without prior separation. For this purpose, a hollow fiber module with a cut-off of 10 kDa and a membrane surface area of 155 cm² was used, through which the coffee extract (mixed with 300 μg/L acrylamide) was circulated at 50° C. On the permeate side of the hollow fiber module, an enzyme solution with amide was added. An enzyme solution with amidase at a concentration of 940 U/L was added to the permeate side of the hollow fiber module. This was not pumped in a circle. The acrylamide diffuses through the membrane and is degraded by the amidase. The acrylamide reduction after a reaction time of 200 minutes was 35%. The acrylamide reduction could be increased to a value of 90% by using a larger membrane area by connecting two modules in series and increasing the enzyme concentration on the permeate side to 8,000 U/L.

### Example 4: Production of Coffee Extract

Raw coffee of the variety Vietnam Robusta was roasted to a color value of 102 scale parts (Neuhaus Neotec, Colortest II). The acrylamide content in the roasted coffee was analyzed and found to be 360 μg/kg. 20 kg of roasted coffee were extracted at an extraction temperature of 110° C., resulting in a total yield of 80 kg of coffee extract. The coffee extract had a dry residue (solids content) of 4.5% and an acrylamide content of 50 μg/L.

### Example 5: Preparation of a CEF (Coffee Extract Filtrate); Permeate (P1)

### Example 5.1: Production of CEF on a Laboratory Scale

500 mL of coffee extract, according to example 4, were filtered in a cross-flow filtration using a capillary module from Repligen with fibers made of mPES, a cut-off of 10 kDa and a filter area of 75 cm² and a permeate (P1) and a retentate (R1) were obtained. The transmembrane pressure was 1.3-1.7 bar. Within 16 h, 400 ml of permeate (P1) was obtained. The experiment took place at room temperature.

### Example 5.2: Production of CEF on a Pilot Scale

### Production with Membrane Module

80 L of coffee extract with 4.5% solids content according to example 4 were filtered in a cross-flow filtration using a membrane module from Microdyn-Nadir with a surface area of 5 m³ (FS10-FC-FUS0181). The membrane material consisted of PES with a cut-off of 10 kDa. The individual fibers had an inner diameter of 0.8 mm. At a temperature of 50° C., an overflow rate of 1 m/s and a TMP of 2 bar, 70 L permeate (P1) (filtered coffee extract) was obtained within 25 min.

### Production with Ceramic Module 61 L of coffee extract with 4.5% solids content according to example 4 were filtered in a cross-flow filtration using a ceramic module from Atech with a surface area of 0.236 m³. The ceramic membrane material has a cut-off of 25 kDa. The individual channels had an internal diameter of 3.3 mm. At a temperature of 50° C., an overflow rate of 5 m/s and a TMP of 1.5 bar, 20.5 L permeate (P1) (filtered coffee extract) was obtained within 48 min.

### Example 6: Enzyme Treatment of Permeate P1, Separation of Biocatalyst

### Example 6.1: Separation of the Biocatalyst via UF

1 L of filtered CEF (permeate (P1)) according to example 5 was mixed with 1 mM acrylamide to simplify the analysis. 1000 U of a liquid amidase preparation was added, mixed and incubated for 2 h at 40° C. The acrylamide concentration decreased by 72% compared to the untreated coffee filtrate (permeate (P1)).

Cross-flow filtration was carried out to separate the enzyme. A membrane module with a cellulose-based membrane with an area of 200 cm² and a pore size of 10 kDa was used for this purpose. The treated CEF was reduced from 1 L to about 50 ml. Approximately 950 ml of treated enzyme-free CEF (permeate (P2)) was obtained.

### Example 6.2: Continuous Enzyme Treatment and Separation of Treated Permeate (P2)

The setup for continuous enzyme treatment consisted of a reaction tank, a receiver tank, a peristaltic pump, a membrane module and a permeate tank, which were connected to each other via hoses. The receiver tank was filled with CEF (permeate (P1) according to example 5), which was enriched with 1 mM acrylamide to simplify the analysis. The reaction container consisted of a vessel into which 75 ml CEF (permeate (P1) according to example 5) was placed and to which 320 U of a liquid amidase preparation was added. The reaction mixture was circulated over the membrane module (200 cm², 10 kDa cellulose-based membrane material) and then pumped back into the reaction vessel. The permeate flow of the membrane module was regulated via the pump capacity and set to a value of 1 ml/min. The permeate was collected in the permeate container. The volume of the reaction mixture was kept constant by pumping filtered coffee extract from the receiver container into the reaction container. The size of the reaction batch and the permeate flow rate resulted in an average retention time for the CEF of 75 min. After equilibration, the permeate showed an acrylamide reduction of 74%.

### Example 6.3: Continuous Enzyme Treatment Using Immobilized Material

A porous enzyme carrier with particle sizes of 300-700 um and pores in the size range of 1200-1800 Å was covalently loaded with enzyme. The specific activity of the resulting enzyme immobilizate was 45 U/g.

5 g of the immobilizate was packed into a chromatography column with a sieve tray and an inner diameter of 1.6 cm. The template with CEF (permeate (P1) according to example 5), enriched with 1 mM acrylamide, and the column filled with immobilizate were tempered to 40° C. CEF (permeate (P1) according to example 5) was continuously passed over the column bed at a flow rate of 2 ml/min and collected fractionated as permeate (P2). The concentration of acrylamide in the column flow-through was reduced by 87%.

### Example 7: Acrylamide Degradation as a Combined 2-Stage Membrane Method

The combined filtration of the coffee extract, the enzymatic treatment with amidase and the separation of the biocatalyst is carried out by interconnecting two cross-flow filtration apparatuses. The receiver vessel 1 of apparatus 1 is filled with 10 L of a coffee extract enriched with acrylamide to 1 mM (permeate (P1) according to example 4). The receiver vessel 1 is connected via a pump to a capillary module 1 from Microdyn-Nadir with 0.25 m² filter surface consisting of a PES membrane with 30 kDa pores. After leaving the capillary module 1, the retentate (R1) is returned to the receiver vessel 1. The permeate outlet of capillary module 1 flows into the receiver vessel 2 of apparatus 2. 2000 U of an amidase preparation are placed in receiver vessel 2. The reservoir vessel 2 is connected via a second pump to a capillary module 2, identical to capillary module 1, whose retentate has been returned to reservoir vessel 2. The permeate outlet of capillary module 2 is connected to reservoir 1 of apparatus 1.

First, apparatus 1 is started with a flow rate of 11 L/min and a TMP of 1.5 bar, whereby filtrate is formed, which flows into the receiver vessel 2. Once the target volume has been reached in the receiver vessel 2, apparatus 2 is started with a flow rate of 5 L/min. The TMP of apparatus 2 is regulated (1-1.5 bar) so that the permeate flow of apparatus 2 is the same as that of apparatus 1 and thus the target volume in receiver vessel 2 remains constant. The acrylamide concentration of the coffee extract in receiver vessel 1 is measured continuously. The acrylamide concentration is reduced by up to 71% after 2 hours. To end the treatment, apparatus 1 is stopped and the volume in apparatus 2 is reduced to the dead volume of 0.4 L in apparatus 2.

### Example 8: Acrylamide Degradation as a Diffusion Membrane Reactor (DMR) Method

### Example 8.1: Batch Method Laboratory Scale

For the direct treatment of coffee extract according to example 4, 1 L of coffee extract according to example 4 are mixed with 1 mM acrylamide and pumped at 40° C. in a circle over a capillary module from Repligen with fibers made of mPES, a cut-off of 10 kDa and a filter area of 75 cm² (membrane (M1)). An amidase enzyme preparation with an activity of 500 U is located on the closed permeate side. The decrease in acrylamide concentration in the coffee extract is monitored over time. After 2 h, the acrylamide content is reduced by 32%.

### Example 8.2: Continuous Method on a Laboratory Scale

For a continuous treatment, the retentate spaces of 3 capillary modules (Repligen, mPES, 10 kDa, 75 cm2) were successively connected via tubes in a further experiment. The permeate spaces of the 3 capillary modules were also connected and filled with 600 U of an amidase preparation. At a temperature of 40° C., coffee extract according to example 4, to which 1 mM acrylamide was added, was pumped at a flow rate of 0.6 ml/min via the retentate side of the capillary modules and collected fractionated. The acrylamide reduction in the coffee extract at the outlet of the 3rd capillary module was 49%.

### Example 8.3: Batch Method on a Pilot Scale

32 L of coffee extract according to example 4 enriched with 3 μM acrylamide were pumped at 50° C. through a membrane module from Microdyn-Nadir with a surface area of 5 m³ at a rate of 5.3 L/min (FS10-FC-FUS0181). The membrane material consisted of PES with a cut-off of 10 kDa. The two outlets of the permeate chamber were connected to a reservoir and filled with 128 kU of a liquid amidase preparation. The liquid was kept in motion by a peristaltic pump. The decrease in acrylamide concentration was monitored over time. After 90 min, the acrylamide concentration was reduced by 92%.

### Example 9: Production of an Acrylamide-Reduced Instant Coffee Product

### Example 9.1: Production Using a Two-Stage Membrane Method

20 kg of coffee of the variety Brazil NY 17/18 were roasted to a color value of 110 (Neuhaus Neotec, Colortest II) and extracted at 110° C. The result was 100 kg of coffee extract with a solids content of 4.1% and an acrylamide content of 50 μg/L.

32 kg of the extract were filtered in a cross-flow filtration system using a ceramic module from Atech with a MWCO of 25 kDa, an overflow rate of 5 m/s and a transmembrane pressure of 3.8 bar at 50° C. 18.8 kg of KEF (permeate (P1)) was obtained, to which 2000 U/L amidase was added and incubated for 1 h at 50° C. Of the original 32 kg of extract, 11 kg of retentate (R1) remained and was set aside. The permeate (P2) obtained after the reaction with the amidase was then subjected to a second cross-flow filtration to separate the enzyme. 16.3 kg of permeate (P2) without biocatalyst was obtained. The treated KEF (permeate (P2)) was combined with 11 kg of the retentate (R1). The resulting mixture of concentrated coffee extract (retentate (R1) from the first cross-flow filtration) and treated coffee extract filtrate (permeate (P2) from the second cross-flow filtration) was transferred to shallow trays, frozen at −40° C. and then freeze-dried in a freeze-drying unit for 29 hours. The result was 140 g of instant coffee product.

Similarly, 3.6 kg of untreated coffee extract, as produced at the beginning of this example, was freeze-dried.

The acrylamide content of the treated and untreated coffee extract was determined. According to the results, the acrylamide content of the treated sample was 29% lower than that of the untreated control sample.

| | | | Acrylamide |
|---|---|---|---|
| Sample | Acrylamide [μg/kg] | Mass loss [%] | reduction [%] |
| Untreated sample | 1100 | 3.1 | — |
| Enzyme-treated | 780 | 3.5 | 29 |
| sample | | | |

### Example 9.2: Production Using a Diffusion Membrane Reactor (DMR)

Raw coffee from an Arabica blend was roasted to a color value of 110. 40 kg of the coffee were extracted at 85° C. A total of 200 kg of coffee extract was obtained. The coffee was then concentrated to a solids content of 12.5% by means of freeze concentration.

The concentrated extract had an acrylamide content of 120 μg/L. The concentrated extract was treated using a diffusion membrane reactor consisting of a cross-flow filtration system with a connected hollow fiber module from Microdyn-Nadir with a filter area of 5 m² and a MWCO of 10 kDa. The permeate chamber (P1) of the module contained 217.6 kU of amidase.

32 L of the extract was pumped unpressurized at 50° C. for 2 h over the hollow fibre module in a circle. Subsequently, 0.6 kg of the treated KEF (permeate (P2)) was transferred to shallow containers, frozen at −40° C. and freeze-dried.

Similarly, 0.6 kg of concentrated extract, which was not subjected to enzyme treatment, was freeze-dried. The acrylamide content of the two instant coffee products was examined and compared. The product made from treated extract has an acrylamide content reduced by 34%.

| | | | Acrylamide |
|---|---|---|---|
| Sample | Acrylamide [μg/kg] | Mass loss [%] | reduction [%] |
| Untreated sample | 750 | 4.53 | — |
| Enzyme-treated | 100 | 4.97 | 87 |
| sample | | | |

1. A method for removing or converting acrylamide from an aqueous preparation to produce a final product having a reduced acrylamide content, comprising the following steps:
(i) providing an aqueous preparation containing acrylamide and other components as a starting product for the production of a final product with a reduced acrylamide content;
(ii) dividing the aqueous preparation into two material streams to obtain a retentate (R1) and a permeate (P1), wherein the retentate (R1) contains the further components (K1), and wherein the permeate (P1) contains acrylamide;
(iii) bringing the permeate (P1) into contact with an acrylamide-reducing biocatalyst and obtaining a permeate (P2) with a reduced acrylamide content;
(iv) combining the obtained permeate (P2) having a reduced content of acrylamide and the retentate (R1) from step (ii) and obtaining an aqueous preparation with having a reduced content of acrylamide, wherein the permeate (P2) does not contain a biocatalyst;
(v) optionally, further processing the aqueous preparation with reduced acrylamide content from step (iv) to obtain a final product.

2. The method of claim 1, wherein the separation of the material streams in step (ii) is performed as filtration.

3. The method of claim 1, wherein dividing into two material streams in step (ii) is carried out as membrane filtration and the pore size of the membrane is d₉₀≤100 kDa.

4. The method of claim 1, wherein the transmembrane pressure is and/or is maintained between 0.01 mbar and 10 bar.

5. The method of claim 1, wherein the end product is an end product intended for pleasure, nutrition or cosmetic purposes.

6. The method of claim 1, wherein the biocatalyst is present as a living microorganism and/or as an inactivated microorganism and/or as a cell lysate and/or as a (partially) purified enzyme and/or as an immobilized enzyme.

7. The method of claim 1, wherein the biocatalyst is present as a (partially) purified enzyme and/or as an immobilized enzyme which is an amidase.

8. The method of claim 1, wherein the biocatalyst is present as a (partially) purified enzyme and/or as an immobilized enzyme which has an amino acid sequence with a sequence identity of at least 60%, to the total length of an enzyme having a sequence selected from the group consisting of the sequences according to SEQ ID NO. 1 to SEQ ID NO. 44, or consists thereof.

9. The method of claim 1, wherein in step (iii) the permeate (P1) is brought into contact with the biocatalyst by mixing and subsequently this mixture is divided into two material streams to obtain the permeate (P2), wherein the permeate (P2) does not contain biocatalyst.

10. The method of claim 1, wherein the permeate (P1) is brought into contact with the biocatalyst via the contact surface of a membrane.

11. The method of claim 1, wherein step (iii) comprises the following substeps:
(iii-1) contacting the permeate (P1) with an acrylamide-reducing biocatalyst and obtaining a permeate (P2) having a reduced content of acrylamide, the permeate (P2) comprising the biocatalyst;
(iii-2) separating the biocatalyst from the permeate (P2) from step (iii-1) by means of separation over a membrane (M2) and obtaining a retentate (R2) containing the biocatalyst and obtaining the permeate (P2) with a reduced content of acrylamide, wherein the permeate (P2) does not comprise any biocatalyst.

12. The method of claim 1, wherein dividing of the aqueous preparation into two material streams in step (ii) and the separation of the biocatalyst before step (iv) are carried out via a single membrane (M1).

13. The method of claim 11, wherein steps (ii) and (iii-2) of the process according to the invention are carried out via a single membrane (M1).

14. The method of claim 11, wherein the pore sizes of the membrane (M1) is smaller than or equal to the pore size of the membrane (M2), and the pore sizes of the membrane (M1) and the membrane (M2) are each smaller than the biocatalyst.

15. The method of claim 11, wherein the pore size of the membrane is d₉₀≤100 kDa.

16. The method of claim 11, wherein the transmembrane pressure is and/or is maintained between 0.01 mbar and 10 bar.

17. The method of claim 1, wherein the aqueous preparation with reduced content of acrylamide obtained in step (iv), compared to the aqueous preparation provided in step (i), has a reduced acrylamide content of at least 20% by weight based on the total weight of the aqueous preparation,
and/or
wherein the aqueous preparation with reduced acrylamide content obtained in step (iv) has an acrylamide content of less than 2000 μg/kg based on the dry mass of the preparation.

18. The method of claim 1, wherein the aqueous preparation obtained in step (iv) is further processed in step (v) and wherein the further processing comprises at least one process step selected from the group consisting of drying, evaporation, concentration, freeze-drying, fluidized bed drying, spray-drying, granulation, comminution and filtration.


SEQUENCE LISTING (44 sequences)

SEQ ID NO: 1 - protein, 513 aa, synthetic construct
  REGION 1..513: note = Description of sequence: Artificial Sequence
MSIHMPDPDAVARMAAEGRFGIPESDLPTYHASVTGLLGSWNKVEELYAEVAPTPPQRSWTRPDAEDNKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 2 - protein, 513 aa, synthetic construct
  REGION 1..513: note = Description of sequence: Artificial Sequence
MSIHMPDPDAVARMAAEGRFGIPESDLPTYHASVTGLLGSWNKVEELYAEVAPTPPQRSWTRPDAEDDKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 3 - protein, 513 aa, synthetic construct
  REGION 1..513: note = Description of sequence: Artificial Sequence
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 4 - protein, 513 aa, synthetic construct
  REGION 1..513: note = Description of sequence: Artificial Sequence
MSIHMPDPDAVARMAAEGRFGIPESDLPTYHAFVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 5 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 6 - protein, 513 aa, synthetic construct
  REGION 1..513: note = Description of sequence: Artificial Sequence
MSIHMPDPDAVARMAAEGRFGIPESDLPTYHASVTGLLGSWNKVEELYAEVAPTPPQRSWTRPDAEDDKLGAWAVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTAPFDVTGHPACSVPAGLADGLPVGLMIVGKHFDDATVLKVANAFEQAVGGFPTPAAERSSA

SEQ ID NO: 7 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDDKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTCPFDVTGHPACSVPAGLADGLPVGLMIVGKHFDDATVLKVANAFEQAVGGFPTPAAERSSA

SEQ ID NO: 8 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTCPFDVTGHPACSVPAGLADGLPVGLMIVGKHFDDATVLKVANAFEQAVGGFPTPAAERSSA

SEQ ID NO: 9 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 10 - protein, 513 aa, synthetic construct
  REGION 1..513: note = Description of sequence: Artificial Sequence
MSIHMPDPDAVARMAAEGRFGIPESDLPTYHASVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTCPFDVTGHPACSVPAGLADGLPVGLMIVGKHFDDATVLKVANAFEQAVGGFPTPAAERSSA

SEQ ID NO: 11 - protein, 513 aa, synthetic construct
  REGION 1..513: note = Description of sequence: Artificial Sequence
MSIHMPDPDAVARMAAEGRFGIPESDLPTYHAHVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAERSSA

SEQ ID NO: 12 - protein, 513 aa, synthetic construct
  REGION 1..513: note = Description of sequence: Artificial Sequence
MSIHMPDPDAVARMAAEGRFGIPESDLPTYHARVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTDPFDVTGHPACSVPAGLADGLPVGLMIVGKHFDDATVLKVANAFEQAVGGFPTPPAERSSA

SEQ ID NO: 13 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTDPFDVTGHPACSVPAGLADGLPVGLMIVGKHFDDATVLKVANAFEQAVGGFPTPPAERSSA

SEQ ID NO: 14 - protein, 513 aa, synthetic construct
  REGION 1..513: note = Description of sequence: Artificial Sequence
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTDPFDVTGHPACSVPAGLADGLPVGLMIVGKHFDDATVLKVANAFEQAVGGFPTPAAERSSA

SEQ ID NO: 15 - protein, 513 aa, synthetic construct
  REGION 1..513: note = Description of sequence: Artificial Sequence
MSIHMPDPDAVARMAAEGRFGIPESDLPTYHARVTGLLGSYNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTAPFDVTGHPACSVPAGLADGLPVGLMIVGKHFDDATVLKVANAFEQAVGGFPTPAAERSSA

SEQ ID NO: 16 - protein, 513 aa, synthetic construct
  REGION 1..513: note = Description of sequence: Artificial Sequence
MSIHMPDPDAVARMAAEGRFGIPESDLPTYHARVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTCPFDVTGHPACSVPAGLADGLPVGLMIVGKHFDDATVLKVANAFEQAVGGFPTPPAERSSA

SEQ ID NO: 17 - protein, 513 aa, synthetic construct
  REGION 1..513: note = Description of sequence: Artificial Sequence
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTNPFDVTGHPACSVPAGLADGLPVGLMIVGKHFDDATVLKVANAFEQAVGGFPTPAAERSSA

SEQ ID NO: 18 - protein, 513 aa, synthetic construct
  REGION 1..513: note = Description of sequence: Artificial Sequence
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTQPFDVTGHPACSVPAGLADGLPVGLMIVGKHFDDATVLKVANAFEQAVGGFPTPAAERSSA

SEQ ID NO: 19 - protein, 513 aa, synthetic construct
  REGION 1..513: note = Description of sequence: Artificial Sequence
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTEPFDVTGHPACSVPAGLADGLPVGLMIVGKHFDDATVLKVANAFEQAVGGFPTPAAERSSA

SEQ ID NO: 20 - protein, 513 aa, synthetic construct
  REGION 1..513: note = Description of sequence: Artificial Sequence
MSIHMPDPDAVARMAAEGRFGIPESDLPTYHAYVTGLLGSYNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEHLANTKPFDVTGHPACSVPAGLADGLPVGLMIVGKHFDDATVLKVANAFEQAVGGFPTPAAERSSA

SEQ ID NO: 21 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTANFDVTGHPACSVPAGLADGLPVGLMIVGKHFDDATVLKVANAFEQAVGGFPTPAAERSSA

SEQ ID NO: 22 - protein, 1026 aa, synthetic construct
  REGION 1..1026: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDGTGHPACSVPAGLADGLPVGLMIVGKHFDDATVLKVANAFEQAVGGFPTPAAERSSAMSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 23 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTVPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 24 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTDPFDVTGHPACSVPAGLADGLPVGLMIVGKHFDDATVLKVANAFEQAVGGFPTPAAERSSA

SEQ ID NO: 25 - protein, 513 aa, synthetic construct
  REGION 1..513: note = Description of sequence: Artificial Sequence
MSIHMPDPDAVARMAAEGRFGIPESDLPTYHAYVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 26 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSAALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 27 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPPAERSSA

SEQ ID NO: 28 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTSPFDVTGHPACSVPAGLADGLPVGLMIVGKHFDDATVLKVANAFEQAVGGFPTPAAERSSA

SEQ ID NO: 29 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAIKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 30 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSIPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 31 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAFCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 32 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEHLANTAPFDVTGHPACSVPAGLADGLPVGLMIVGKHFDDATVLKVANAFEQAVGGFPTPAAERSSA

SEQ ID NO: 33 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTRPFDVTGHPACSVPAGLADGLPVGLMIVGKHFDDATVLKVANAFEQAVGGFPTPAAERSSA

SEQ ID NO: 34 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFGIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 35 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYRGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 36 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWRVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 37 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARSLEMLANTAPFDVTGHPACSVPAGLADGLPVGLMIVGKHFDDATVLKVANAFEQAVGGFPTPAAERSSA

SEQ ID NO: 38 - protein, 513 aa, synthetic construct
  REGION 1..513: note = Description of sequence: Artificial Sequence
MSIHMPDPDAVARMAAEGRFGIPESDLPTYHARVTGLLGSWNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHCGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 39 - protein, 513 aa, synthetic construct
  REGION 1..513: note = Description of sequence: Artificial Sequence
MSIHMPDPDAVARMAAEGRFGIPESDLPTYHARVTGLLGSYNKVEELYAEVAPTPPQRSWTRPDAEDNKLGAWYVQTSITETSEGPLAGRTVAVKDNVAVAGVPMMNGSRTLEGFVPAEDATVVSRLLAAGATIAGKSVCEDLCFSGGSHTSKPGPVHNPWDMSRSAGGSSSGSGALVAAGEVDMAIGGDQGGSIRIPSAYCGTVGHKPTHGLVPYTGGFPIEQTIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVIATEGGLWQMVEGNAYGMNWKGHYDPSLIAYYGRKWREDPAQFSETVKLVALAGRYALTTQYGRHYAMARNLAPKLVAAYDAALSNYDVLVMPTLPMRATVLPGPDAPVEEILARALEMLANTAPFDVTGHPACSVPAGLADGLPVGLMIVGKHFDDATVLKVANAFEQAVGGFPTPAAERSSA

SEQ ID NO: 40 - protein, 348 aa, Parageobacillus thermoglucosidasius
  REGION 1..348: note = Description of sequence: Parageobacillus thermoglucosidasius Sequence
MRHGDISSSHDTVGVAVVNYKMPRLHTKKEVIENAKNIANMIVGMKQGLPGMDLVIFPEYSTMGIMYDRKEMFETATTIPGPETEIFAEACRKANTWGVFSLTGEQHEEHPHKNPYNTLVLINNKGEIVQKYRKIIPWCPIEGWYPGDTTYVTEGPKGIKISLIICDDGNYPEIWRDCAMKGAELIVRCQGYMYPAKEQQIMMAKTMAWANNVYVAVANATGFDGVYSYFGHSAIIGFDGRTLGECGEEENGIQYAEISLSQIRDFRKNAQSQNHLFKLLHRGYTGIIXSGEGDKGVAECPFDFYRTWVMDAEKARENVEKITRTTIGTAECPIEGIPHEGKEKEASV

SEQ ID NO: 41 - protein, 348 aa, Aeribacillus pallidus
  REGION 1..348: note = Description of sequence: Strain RAPc8
MRHGDISSSHDTVGIAVVNYKMPRLHTKAEVIENAKKIADMVVGMKQGLPGMDLVVFPEYSTMGIMYDQDEMFATAASIPGEETAIFAEACKKADTWGVFSLTGEKHEDHPNKAPYNTLVLINNKGEIVQKYRKIIPWCPIEGWYPGDTTYVTEGPKGLKISLIVCDDGNYPEIWRDCAMKGAELIVRCQGYMYPAKEQQIMMAKAMAWANNTYVAVANATGFDGVYSYFGHSAIIGFDGRTLGECGTEENGIQYAEVSISQIRDFRKNAQSQNHLFKLLHRGYTGLINSGEGDRGVAECPFDFYRTWVLDAEKARENVEKITRSTVGTAECPIQGIPNEGKTKEIGV

SEQ ID NO: 42 - protein, 348 aa, Geobacillus stearothermophilus
  REGION 1..348: note = Description of sequence: Geobacillus stearothermophilus Sequence
MRHGDISSSHDTVGVAVVNYKMPRLHTKKEVIENAKNIANMIVGMKQGLPGMDLVIFPEYSTMGIMYDRKEMFETATTIPGPETEIFAEACRKANTWGVFSLTGEQHEEHPHKNPYNTLVLINNKGEIVQKYRKIIPWCPIEGWYPGDTTYVTEGPKGIKISLIICDDGNYPEIWRDCAMKGAELIVRCQGYMYPAKEQQIMMAKTMAWANNVYVAVANATGFDGVYSYFGHSAIIGFDGRTLGECGEEENGIQYAEISLSQIRDFRQNAQSQNHLFKLLHRGYTGIIQSGEGDKGVAECPFDFYRTWVMDAEKARENVEKITRTTIGTAECPIEGIPHEGKEKEASV

SEQ ID NO: 43 - protein, 346 aa, Pseudomonas aeruginosa [sequence omitted]

SEQ ID NO: 44 - protein, 343 aa, Methylophilus methylotrophus
  REGION 1..343: note = Description of sequence: Methylophilus methylotrophus Sequence
MIHGDISSSQDTVGVAVVNYKMPRLHTKAEILANCRNIADMVVNMKAGLPGMDLVIFPEYSTHGIMYDSQEMYDNATTVPGPETDIFAEACRKAKVWGVFSITGEQHEEHPKKAPYNTLILMNDQGEIVQKYRKIMPWVPIEGWYPGKETFISEGPKGLKVSLIICDDGNYPEIWRDCAMKGAELIVRCQGYMYPAKEQQILISKAMAFANNTYVAVSNAAGFDGVYSYFGHSAIIGFDGRTLGECGEEENGVQYAALSKHLIRDFRKHGQSENHLFKLLHRGYTGMLNSGDGDQGVATCPYSFYSKWVQDPAAARDMVESFTRTTLGTKEAPIAGLPNEEND